(19) United States
(12) Patent Application Publication
(10) Pub. No.: US 2023/0314424 A1
(43) Pub. Date: Oct. 5, 2023

(54) FLUID TESTING DEVICE

(71) Applicant: RJS MEDIAGNOSTIX, New York, NY (US)

(72) Inventors: Balwant Rai, Roedovre (DK); Jasdeep Kaur, Roedovre (DE)

(21) Appl. No.: 18/041,175

(22) PCT Filed: Aug. 12, 2021
(86) PCT No.: PCT/IB2021/057454

§ 371 Date: Feb. 9, 2023

Related U.S. Application Data
(60) Provisional application No. 63/066,584, filed on Aug. 17, 2020.

(51) Int. Cl. G01N 33/543; G01N 21/78; G01N 33/58

(52) U.S. Cl. CPC G01N 33/54388; G01N 21/78; G01N 33/587; G01N 2021/7759

(57) ABSTRACT

Testing devices are provided for detecting the level of one or more analytes in a fluid. In an example, a testing device includes a fluid collection device adapted to collect a fluid, and an assembly including a filter coupled to a test matrix, the test matrix including one or more lateral flow strips adapted to optically indicate a level of one or more analytes in the fluid, where the fluid collection device is configured to supply the fluid to the one or more lateral flow strips via the filter.

### CROSS-REFERENCE TO RELATED APPLICATIONS

The present application claims priority to U.S. Provisional Application No. 63/066,584 entitled “FLUID TESTING DEVICE”, and filed on Aug. 17, 2020. The entire contents of the above-listed application are hereby incorporated by reference for all purposes.

### FIELD OF TECHNOLOGY

The present description relates generally to a testing device, specifically a fluid collection device.

### BACKGROUND

Sampling and testing of biological samples and body fluids is common for both testing and monitoring for any number of biochemical or physiological conditions. Such testing is often performed at the point-of-care (POC), e.g., at or near the site of patient care, which may increase convenience and the likelihood that the patient, physician, and care team may receive the test results more quickly, which allows for immediate clinical management decisions to be made. POC testing devices may often rely on lateral-flow chromatographic immunoassay cassettes. Lateral-flow chromatographic immunoassay cassettes can be used to easily and quickly obtain a variety of qualitative results relating to a number of biochemical and physiological conditions and disease states.

Lateral flow chromatographic immunoassay test cassettes are easy to administer, have a broad applicability to a variety of analytes, and can be deployed in locations where medical testing laboratories are not readily available, and thus provide a great benefit for the diagnosis and control of disease in individuals including humans. However, current lateral flow chromatographic immunoassay tests may suffer from sample contamination if care is not taken when collecting the fluid that will be tested. Further, the collection of the fluid and application to the lateral flow chromatographic immunoassay test cassette may be challenging for some fluids where fluid volume is limited and/or difficult to access (e.g., saliva), which may lead to compromised test results if sufficient fluid is not collected, fluid is lost upon application to the lateral flow chromatographic immunoassay test cassette, etc.

### SUMMARY

Embodiments of a testing device are provided herein. In one example, a testing device includes a fluid collection device adapted to collect a fluid, and an assembly including a filter coupled to a test matrix, the test matrix including one or more lateral flow strips adapted to optically indicate a level of one or more analytes in the fluid, where the fluid collection device is configured to supply the fluid to the one or more lateral flow strips via the filter.

The testing device may provide rapid and fast results due to the testing device being configured to simultaneously collect a sample, filter the sample, and analyze the biomarkers in the sample. In some examples, pressure from the cap may be applied to facilitate the fast collecting, filtering, and testing the biomarkers of the sample.

To the accomplishment of the foregoing and related ends, certain illustrative aspects of the system are described herein in connection with the following description and the attached drawings. The features, functions, and advantages that have been discussed can be achieved independently in various embodiments of the present disclosure or may be combined in yet other embodiments, further details of which can be seen with reference to the following description and drawings. The summary is provided to introduce a selection of concepts in a simplified form that are further described below in the Detailed Description. This summary is not intended to identify key features or essential features of any subject matter described herein.

### BRIEF DESCRIPTION OF THE DRAWINGS

FIG. 1 schematically shows a first side view of a testing device according to an embodiment of the disclosure.

FIG. 2 schematically shows a second side view of the testing device of FIG. 1.

FIG. 3 schematically shows a top view of the testing device of FIG. 1.

FIG. 4 schematically shows a perspective view of the testing device of FIG. 1.

FIG. 5 schematically shows a testing device according to another embodiment of the disclosure.

FIG. 6 shows an example funnel-shaped fluid collection device of a cap of a testing device according to the disclosure.

FIG. 7 schematically shows a testing device according to another embodiment of the disclosure.

FIG. 8 shows an example testing matrix that may be included in the testing device of FIG. 1.

FIG. 9 is a flowchart illustrating an example method for analyzing images of the testing matrix of the testing device of FIG. 1.

FIG. 10 is a graph showing signal intensities for detection of a biomarker using two wavelengths of light.

### DETAILED DESCRIPTION

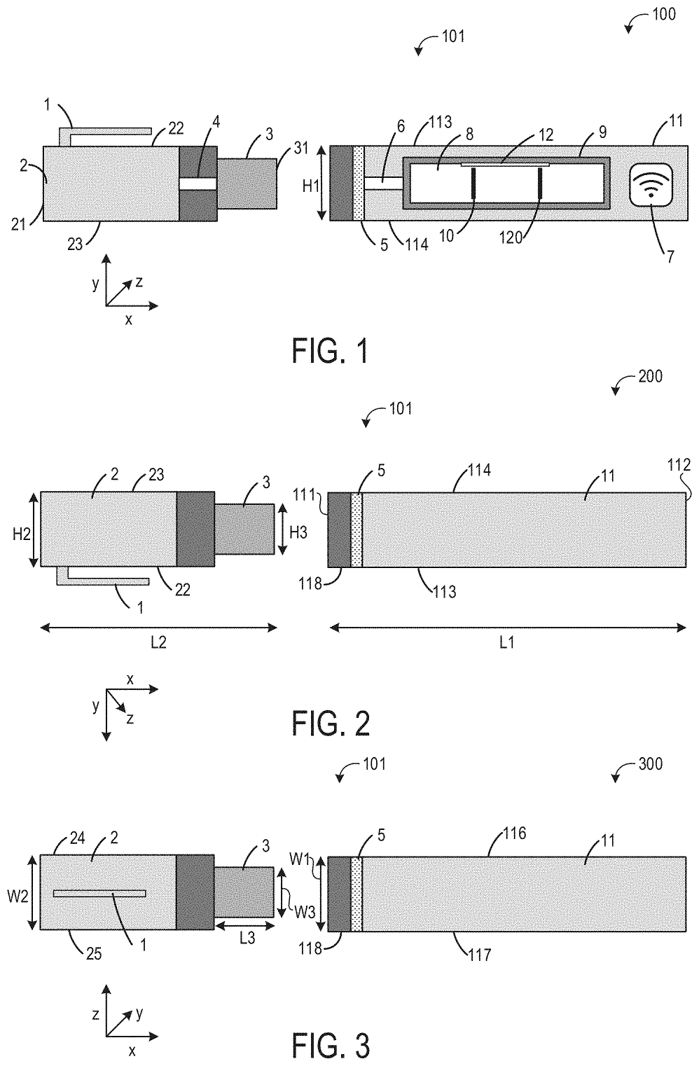

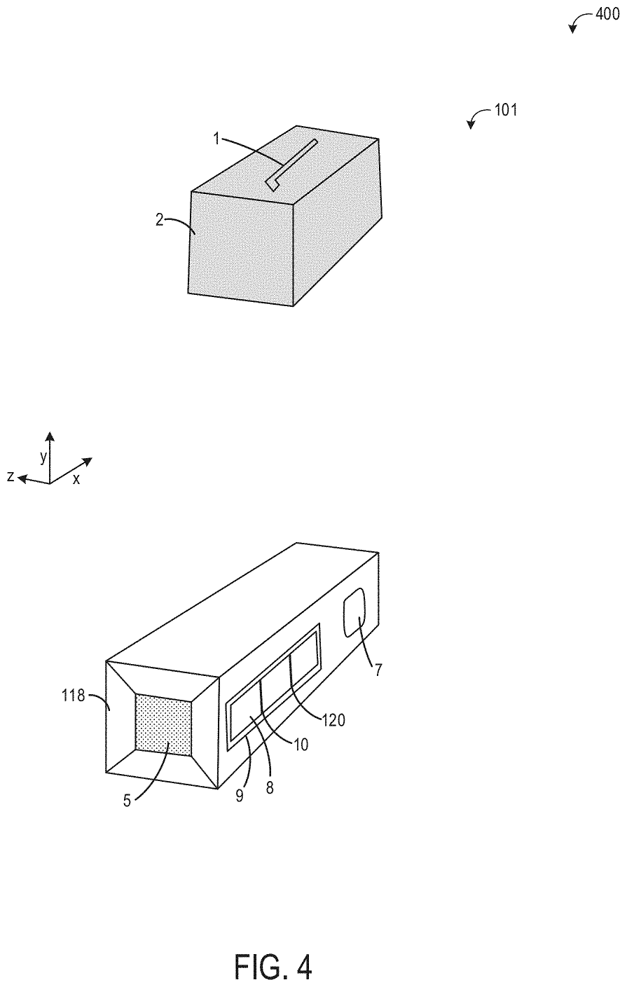

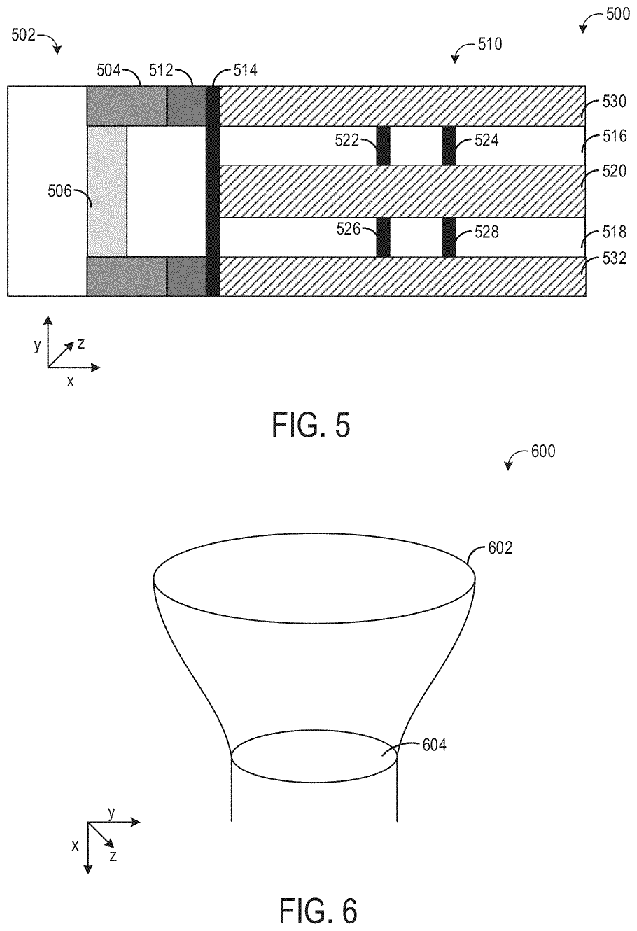

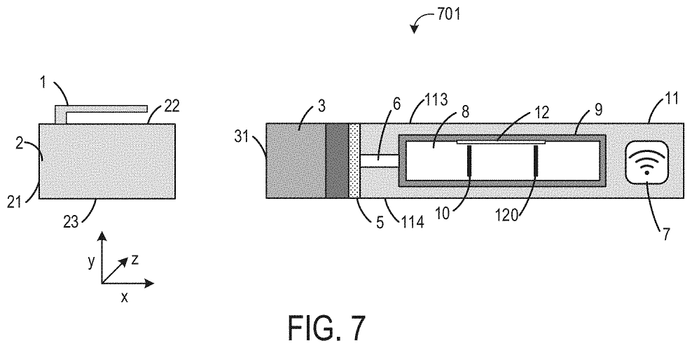

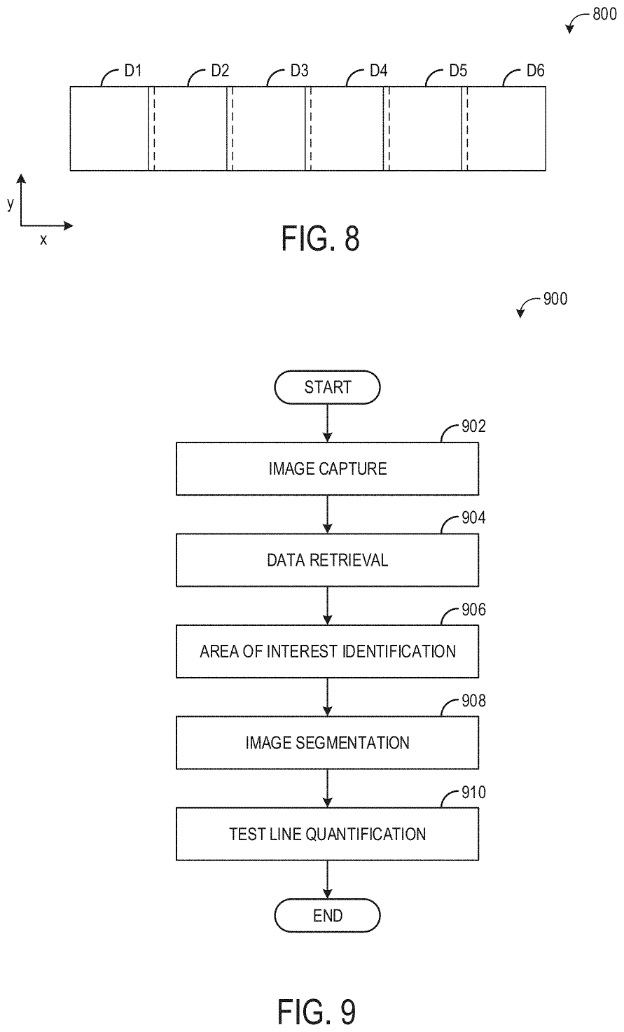

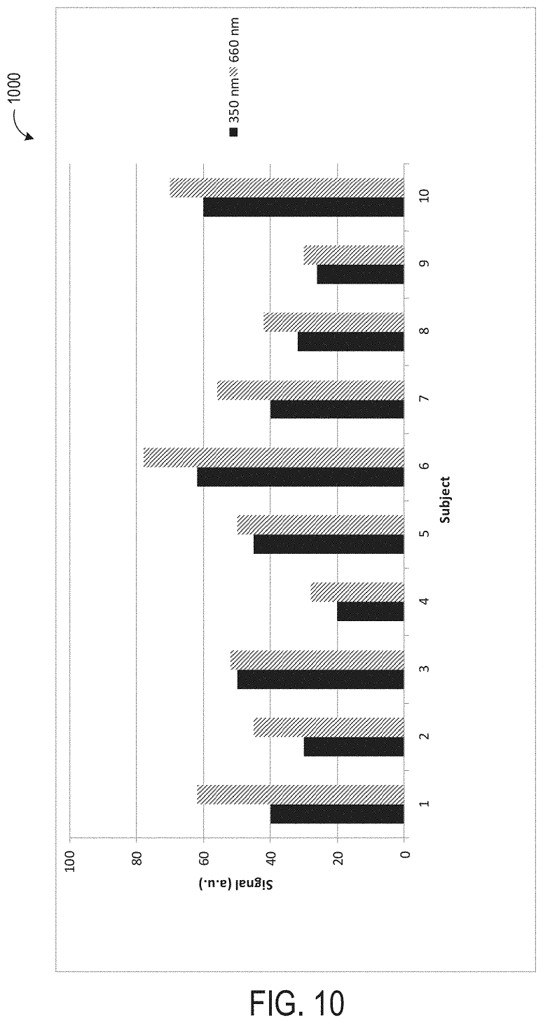

FIGS. 1-4 show an example testing device 101 according to the disclosure. FIG. 1 shows a first side view 100 of the testing device 101 (e.g., a front view), FIG. 2 shows a second side view 200 of the testing device 101 (e.g., a back view), FIG. 3 shows a top view 300 of the testing device 101, and FIG. 4 shows a perspective view 400 of the testing device 101. FIGS. 1-4 will be described collectively. FIG. 5 shows a testing device 500 according to another embodiment of the disclosure. FIG. 6 shows a collection funnel which may be attached to or incorporated with a testing device such as testing device 101 or testing device 500. FIG. 7 shows a testing device 501 according to another embodiment of the disclosure. FIG. 8 shows an example testing matrix that may be included in the testing device 101, testing device 500, or testing device 701. Each of FIGS. 1-8 includes a Cartesian coordinate system. FIG. 9 shows an example method for generating test results from the testing device by using an image analysis system. FIG. 10 is a graph showing signal intensities for detection of a biomarker using two different wavelengths of light.

The testing device 101 includes a cap 2 and an assembly 11. The cap 2 includes a handle 1 and a fluid collection device 3. The fluid collection device 3 may be configured to collect fluid, such as saliva, sweat, blood, sputum, cerebral spinal fluid, pleural fluid, milk, lymph, semen, urine, stool, tears, needle aspirate, external section of the skin, respiratory, intestinal, or genitourinary tract, tumor, organ, cell culture, cell culture constituent, tissue sample, tissue section, whole cell, cell constituent, cytospin, cell smear, etc., for application to the assembly 11 in order to test the level of one or more analytes in the fluid, as will be described below. The assembly 11 may include a filter 5, a fluid channel 6, and a test matrix 8, all housed within the assembly 11. The assembly may have a length L1 that extends along the x axis of the coordinate system from a collection end 111 of the assembly 11 to a back end 112 of the assembly, a height H1 that extends along the y axis from a top surface 113 to a bottom surface 114 of the assembly 11, and a width W1 that extends along the z axis from a first side surface 116 to a second side surface 117 of the assembly. In some examples, the L1 of assembly 11 may be in a range of 7-9 inches (e.g., 175-230 mm), such as 8.0 inches (e.g., 203 mm), the width W1 may be in a range of 1-1.5 inches (e.g., 25-40 mm), such as 1.2 inches (e.g., 50 mm), and the height H1 may be in a range of 1-1.5 inches, such as 1.0 inch. In another example, the assembly may have a length L1 of 9.0 inches, a width W1 of 1.2 inches, and a height H1 of 1.5 inches. However, other dimensions are possible without departing from the scope of this disclosure.

The cap 2 may have an overall length L2 and a height H2 (1.2-1.6 inches, such as about 1.2 inches) at a second end 21 of the cap 2, opposite a first end 31 of the fluid collection device 3. The overall length L2 (3-7 inches, such as about 5.0 inches) may extend from the second end 21 to the first end 31 along the x axis of the coordinate system. The height H2 may extend along the y axis, from a top surface 22 of the cap 2 to a bottom surface 23 of the cap 2. Further, the cap 2 may have a width W2 that extends from a first side surface 24 of the cap 2 to a second side surface 25 of the cap 2 along the z axis. The overall length L2 of the cap 2 may be less than the length L1 of the assembly 11, such as about 60-70% of the length L1. In some examples, the height H2 may be the same as the height H1 and the width W2 may be the same as the width W1.

The fluid collection device 3 may extend outward from the main body of the cap 2 with a length (L3). The fluid collection device 3 may have a smaller height (H3) (0.8-1.2 inches, such as about 0.9 inches) than the height H2 of the cap 2 and a smaller width (W3) (0.7-1.0 inches, such as about 0.7 inches) than the width W2 of the cap 2. In some examples, the length L3 of the fluid collection device 3 that extends outward from the main body of the cap 2 may be 25% of the overall length L2 of the cap 2, though other relative dimensions are possible without departing from the scope of this disclosure. The height H3 of the fluid collection device 3 may be 70% of the height H2 of the cap and the width W3 of the fluid collection device 3 may be 70% of the width W2 of the cap 2, though other relative dimensions are possible.

The fluid collection device 3 may have a hollow interior and may be open at the first end 31 of the fluid collection device 3. The hollow interior of the fluid collection device 3 may extend at least partially into the interior of the cap 2, at least in some examples. The fluid collection device 3 may be adapted to collect and store fluid in a suitable manner. For example, when the fluid being collected via the fluid collection device 3 is saliva, the cap 2 may be placed into a suitable position near, touching, and/or within the mouth of an individual to allow saliva to be collected in the fluid collection device. For example, the fluid collection device 3 may be positioned against the lower lip of the individual with the first end 31 facing up. The individual may be seated in a bent forward position with their tongue on the lingual surfaces of the upper incisors, which may allow the saliva to drip into the fluid collection device 3. The fluid collection device 3 may be held in this position until a sufficient amount of fluid is collected, e.g., about 100 μL-10 mL of saliva. In some examples, the fluid collection device 3 may be coated with a saliva secretion stimulator that is configured to stimulate the individual's mouth through saliva secretion stimuli. The saliva in the body accelerates secretion, shortens the collection time, and the filter filters the bubbles, food particles and impurities in the collected saliva to ensure that high-quality saliva can be collected. In some examples, the fluid collection device 3 may include a sample compressor with no molecule which specifically binds an analyte. The sample compressor may include a pad and a mobile control binding partner on the pad. In some examples, samples that are collected via the fluid collection device are not diluted prior to application to the assembly. There may be no pretreatment of the sample, and the sample may be collected and transferred to the assembly without any treatment of the collected sample.

The cap 2 may further include a sensor 4. The sensor 4 may be an indicator that indicates to a user of the testing device when sufficient fluid has been collected in the fluid collection device 3. Additionally or alternatively, the sensor 4 may sense fluorescent components of saliva by using special fluorescent dyes e.g., a fluorescent line may appear after saliva reacts with the fluorescent dye. In some examples, the cap 2 and/or fluid collection device 3 may include a pad to facilitate collection of the fluid. In some aspects, the pad may include an indicator (e.g., the sensor 4) to indicate when sufficient fluid has been collected.

The cap 2 may be removably couplable to assembly 11. For example, the cap 2 may be separated from assembly 11 in order to allow for fluid collection in the fluid collection device 3. The cap 2 may then be coupled to/brought into face-sharing contact with the collection end 111 of the assembly 11 in order to supply the collected fluid to the assembly 11. At the collection end 111, the assembly 11 includes the filter 5 fluidly coupled to the fluid channel 6. The fluid channel 6 may fluidly couple the filter 5 to the test matrix 8 in order to allow fluid passing through filter 5 (e.g., from fluid collection device 3) to reach and flow across the test matrix 8. The filter 5 may be a micropore filter having a suitable pore size to filter bacteria and other particles from the fluid, such as membranes including, but not limited to, nylon, polyesters, cellulose, rayon, calcium alginate, polypropylene (PP), polyvinyl difluoride (PVDF), and polytetrafluorethylene (PTFE), etc., and may have pore sizes of about 0.01 μm to 2000 nm, such as about 0.45 In some examples, the filter 5 may be comprised of nitrocellulose or other suitable material.

In some examples, the collection end 111 may include a sealing interface 118 extending along the outer edges of the filter 5. As visualized in FIG. 4, the sealing interface 118 may extend out from the filter 5 a suitable distance and may angle back inward to the filter 5, thereby creating a frame for the filter 5. The exposed portion of the filter 5 (shown in FIG. 4) may have similar dimensions to the fluid collection device 3, and the sealing interface 118 may assist in creating a tighter fluidic coupling between the fluid collection device 3 and the filter 5, when the cap 2 is brought into connection with the assembly 11. In this way, the cap 2 may be separated from the assembly 11 to allow comfortable and easy fluid collection via the fluid collection device 3 of the cap 2. Then, the cap 2 may be brought into face-sharing contact with the collection end 111 of the assembly, allowing the fluid in the fluid connection device 3 to flow into the assembly 11. The sealing interface 118 may ensure the fluid is directed to the fluid channel 6 (via the filter 5) and may reduce loss of the fluid.

The fluid channel 6 may be a hollow passage or conduit within the assembly 11 that is sized to supply the fluid (after passing through the filter 5) to the testing matrix 8 at a target flow rate. In other examples, as shown in FIG. 5, the fluid channel may be dispensed with and fluid may flow directly from the filter to the test matrix. In some aspects, the fluid channel may divide the sample and provide sufficient fluid to each portion (e.g., lateral flow strip) of the test matrix. The sample may be divided equally or unequally depending on the biomarker(s) being tested and/or other clinical requirements.

In some examples, the cap 2 may include a mechanism that, when actuated, causes an increased amount of pressure (e.g., above atmospheric pressure) to be applied to the fluid, filter, fluid channel, etc., in the assembly 11. The mechanism may include a semi-flexible material that may be squeezed by an operator to apply pressure to the assembly once the cap 2 has been coupled to the assembly. In other aspects, other suitable mechanisms such as a plunger may be used. In some aspects, a combination of mechanisms may be used. The amount of pressure applied by actuating the cap 2 may be about 20-70 psi, 30-60 psi, or 40 psi, in an axial (e.g., longitudinal) direction (along the x axis in FIG. 1). The application of pressure may increase the filter rate of the saliva or other bodily fluid, which leads to a faster reaction between the biomarkers in the fluid and the antibody(s) or binding agent(s) in a test matrix.

The fluid channel 6 is fluidly coupled to the test matrix 8, and fluid flowing through the fluid channel 6 is configured to flow over the test matrix 8. The test matrix 8 may include one or more lateral flow strips housed together in a single housing or cassette, at least in some examples. Each lateral flow strip may be about 3-10 mm wide and about 12-20 mm long, in some examples, and may be comprised of a plurality of pads/membranes, such as a sample pad, a conjugate pad, a nitrocellulose membrane (or other suitable membrane), and an absorption pad, mounted in an overlapped fashion on a backing. The sample pad may have a suitable area, such as about 0.6-2.0 cm×0.9-2.5 cm and may be comprised of material (e.g., glass fiber paper) configured to absorb the fluid and flow the fluid (e.g., using capillary flow, wicking, or wetting) to the conjugate pad. The conjugates may include, but are not limited to, colloidal gold, colored latex beads, fluorescent nanoparticles, chemiluminescent nanoparticles, paramagnetic nanoparticles, or phosphorescent nanoparticles and silver, and other suitable agents, and the enhancement element may include at least one sliver salt or gold salt. In some examples, the sample pad may include a buffer or may include a reservoir including the buffer, where the buffer is adapted to facilitate one or more chemical reactions occurring on the lateral flow strip, as described below. The conjugate pad may have an area in a range of about 0.4-0.8 cm×0.4-0.8 cm, in some examples, and may likewise be comprised of a material configured to flow the fluid using capillary flow, wicking, or wetting. The conjugate pad further includes a layer or layers of conjugate within a salt-sugar matrix, for example. With this device, if the conjugate zone on the sample collection device is not adequately compressed and made to directly contact the test strip, no control zone will develop even with a proper flow of the running buffer. Thus, the appearance of the control zone with both the negative and positive test samples indicates a true practical control in the test.

The conjugate may include one or more binding agents, such as, but not limited to, antibodies, peptides, enzymes, and/or other suitable agents, each coupled to a colormetric label (e.g., 10-40 μL of label-agent conjugate). In some aspects, each agent may be specific to an analyte of interest. The analytes of interest may be the same or different. The colormetric label may be a suitable colormetric label, such as gold nanoparticles (e.g., having a diameter of about 10-200 nm), silver nanoparticles, or latex. In some examples, the conjugate pad may include additional reagents that may help facilitate binding between the agents of the conjugate and the analyte of interest and/or help facilitate binding between the agents immobilized on the nitrocellulose or other suitable membrane and the analyte of interest, as described below.

When the fluid flows across the conjugate pad (e.g., for a suitable duration, such as about 1 mintue-20 minutes, for example about 2 minutes), the conjugates are made freely available to flow with the fluid, e.g., the fluid may at least partially dissolve the salt-sugar matrix. If one or more analytes of interest are present in the fluid, the analyte(s) may bind corresponding conjugates. For example, if the testing device is configured to test for the presence of pepsin in saliva, the conjugate may include anti-pepsin antibodies coupled to colormetric labels. When saliva is flowed over the sample pad and then over the conjugate pad, the conjugates including the anti-pepsin antibodies may bind to any pepsin present in the saliva. Free, unbound conjugates as well as conjugates that have bound to an analyte of interest may flow with the fluid to the nitrocellulose membrane.

The nitrocellulose membrane may include one or more test lines, such as test line 10, and a control line 120. Each test line and the control line may have a suitable area, such as about 5-10 mm², may be spaced apart by a suitable distance (e.g., about 1-1.5 cm), and may be comprised of a suitable binding agent (e.g., antibody or antibodies, peptides, enzymes, etc.) immobilized on the membrane. For example, each test line may include a primary antibody specific to an analyte of interest (which is the same analyte(s) of interest as described above) and the control line may include a secondary antibody. In some examples, the test line may include about 0.5-2.5 μL of monoclonal or polyclonal antibody at a concentration of about 40-300 μg/mL in PBS and the control line may include about 0.5-2.5 μL of secondary antibody such as goat, mouse anti-rabbit IgG antibody at a concentration of about 60-90 μg/mL in PBS. In the example presented above where the testing device is configured to test for the presence of pepsin in saliva, the test line 10 may include immobilized anti-pepsin antibodies and the control line 120 may include immobilized secondary antibodies configured to bind to the primary antibody of the conjugate, such as anti-IgG antibodies specific to the species of the primary antibody (e.g., the primary antibody of the conjugate may be rabbit anti-human pepsin antibodies and the secondary antibody may be mouse or goat anti-rabbit IgG antibodies). As the fluid flows over the nitrocellulose membrane, conjugate that has bound the analyte of interest (e.g., pepsin) may bind to the primary antibodies of the test line, while bound or unbound conjugates may bind to the secondary antibodies of the control line. The fluid may continue to flow to the absorption pad, which may collect the fluid. The absorption pad may have the same area as the sample pad, at least in some examples. Different methods for manufacturing different portions of the strip include, but are not limited to, striping, spraying, soaking, and drying materials onto the strip.

The results of the test matrix 8 may be viewable through a window 9 of the assembly. For example, the test matrix 8 may be housed within the assembly 11 and a top surface of the test matrix 8 (e.g., the side opposite the backing) may be visible through the window 9. As the fluid flows over the lateral flow strip, the colormetric labeled conjugates may collect at the control line, causing the control line to turn a certain color (e.g., red if the colormetric label is gold nanoparticles, blue if the colormetric label is latex) to indicate that the test was performed properly (e.g., that the fluid was able to free the conjugates and pass the conjugates over the control line and test line). If the analyte of interest is present in the fluid, the test line will also turn the certain color, due to the collection of the analyte-bound conjugates at the test line. If the analyte of interest is not present in the fluid, the test line will not change to the certain color. Analyte concentrations as low as about 0.01 picogram of analyte per 1 milliliter fluid may be visualized within 1-5 minutes. Further, the amount of fluid that flows via the lateral flow strip may be the same or different than the amount of fluid collected via the fluid collection device 3. In some examples, the amount of fluid that flows over a lateral flow strip may be about 0.1 μL to 200 μL, such as about 0.2 μL to about 10 μL or about 0.5 μL to about 10 μL, about 5 μL, about 10 μL, or about 60 μL or any fraction thereof.

In some examples, the assembly 11 further includes a near field communication (NFC) transmission device 7. The NFC transmission device 7 may be configured to transmit information to an NFC reader, for example, which may be usable by the NFC reader to verify a serial number or other identifying number of the testing device 101. While an NFC transmission device is shown, it is to be understood that testing device 101 may include an alternative communicating device, such as an RFID tag or a BLUETOOTH device. In some examples, the NFC transmission device 7 may be configured to send a QR code that identifies the specific testing device, or a QR code may be printed on the testing device. Such QR codes may be used for any purpose, in some aspects the QR codes may include patient information and serve for identification purposes.

The testing device 101 described herein may be used to test for the presence of a variety of analytes in a fluid, such as pathogens (e.g., viruses or microorganisms such as bacteria, fungi, or parasites), biomarkers such as antibodies, growth factors, complement, cytokines, lymphokines, chemokines, interferons and interferon derivatives, C-reactive protein, calcitonin, amyloid, adhesion molecules, and chemo-attractant components, different molecular, drug, or chemical molecules or complexes and metabolites, other biomarkers of diseases or conditions, etc. As an example, the testing device 101 may be adapted to diagnose and/or monitor treatment of gastroesophageal reflux disease (GERD), reflux laryngitis (RL), laryngopharyngeal reflux disease (LPR), and/or nonerosive reflux disease (NERD) by detecting the presence, absence, level, and/or change in amounts of one or more of salivary biomarkers, including E-cadherin, transforming growth factor-alpha (TGF-α), epidermal growth factor (EGF), interleukin-6 (IL-6), matrix metalloproteinase-2 (MMP-2), matrix metalloproteinase-7 (MMP-7), and pepsin, as well as other genomic, proteomic and metabolic biomarkers. The testing device 101 in such an example may include one or more lateral flow strips as described above including one or more test lines respectively comprising immobilized anti-E-cadherin, anti-TGF-α, anti-IL-6, anti-MMP-2, anti-MMP-7 and/or anti-pepsin antibodies. For example, the testing device 101 may include a first lateral flow strip having a test line comprising anti-E-cadherin antibodies and a second lateral flow strip having a test line comprising anti-EGF antibodies. In other aspects, the testing device 101 may include a first lateral flow strip with multiple testing lines, for example test lines for both anti-E-cadherin antibodies and anti-EGF antibodies. An elevated level of E-cadherin in saliva of a patient along with a decreased level of EGF in the saliva of the patient, as determined via the testing device 101, may be indicative of GERD, LPR, RL and/or NERD. Other combinations of the saliva-based biomarkers that may be included in examples of the testing device 101 to diagnose and/or monitor GERD, LPR, RL, and/or NERD may include a combination of E-cadherin and MMP-7, a combination of E-cadherin and pepsin, a combination of EGF and pepsin, and a combination of pepsin and MMP-7.

In some examples, the testing device 101 may be adapted to provide a quantitative or semi-quantitative analysis of the level of one or more analytes in a fluid. For example, varying levels of bound antibody may be indicated by different points along the test strip or in a series of test lines. For example, a series of test lines capturing differing amounts of analytes could form a gradient along the test strip. In another example, the concentration of one or more analytes may be measured by measuring the color, intensity, and/or brightness of each test line using a special device such as a reader, a camera (e.g., which may be included as part of a mobile computing device such as a smart phone), etc. In some examples, the testing device 101 may include a data storage system and communication subsystem (e.g., the NFC device described above) to allow for communication between the testing device 101, a reader used to measure the color, intensity, and/or brightness of each test and control line, and/or a computing device such as a smartphone, tablet, computer etc. The computing device may execute an application adapted to receive the measurements (whether from the reader or from the testing device), generate a testing result from the measurements, and display the testing result to the user. In some examples, the reader may be integrated in the testing device 101. In other examples, the reader may be a separate device.

As another example, the testing device 101 may be adapted to detect analytes that may include, but are not limited to antibodies to infectious agents (such as HIV, HTLV, Helicobacter pylori, hepatitis, measles, mumps, or rubella), cocaine, benzoylecgonine, benzodizazpine, tetrahydrocannabinol, nicotine, ethanol theophylline, phenytoin, acetaminophen, lithium, diazepam, nortryptyline, secobarbital, phenobarbitol, methamphetamine, theophylline, testosterone, estradiol, estriol, 17-hydroxyprogesterone, progesterone, thyroxine, thyroid stimulating hormone, follicle stimulating hormone, luteinizing hormone, transforming growth factor alpha, epidermal growth factor, insulin-like growth factor I and II, growth hormone release inhibiting factor, IGA and sex hormone binding globulin; and other analytes including antibiotics (e.g., penicillin), glucose, cholesterol, caffeine, cotinine, corticosteroid binding globulin, PSA, or DHEA binding glycoprotein.

Further, while the testing device 101 has been described herein as including a sandwich-type immunochromatographic lateral flow strip with colormetric labels that can be visualized with visible light, other types of testing matrices are possible without departing from the scope of this disclosure, such as competitive-type immunoassays and/or immunoassays where the colormetric labels are visualized with other types of light, such as ultraviolet (UV) light, infrared (IR) light, and/or alternate light imaging (ALI). In some examples, the light source that may be used to facilitate the optical indication of the level of the one or more analytes via the test matrix described herein may be an integrated light source, included in the cap 2 or as part of the assembly 11. For example, a light source 12 may be positioned under the window 9 and above the test matrix 8 along an edge of the test matrix 8 and may direct light at the test matrix 8, such as toward the center of the test matrix 8 and/or toward the test line 10 and control line 120. The integrated light source may include one or more of a UV light source, an IR light source, an ALI light source, or a visible light source. When included, the integrated light source may be connected to a circuit board, and the circuit board may be connected to and/or include a switch and an internal or external power source, such that the light source may be powered on and off as needed (e.g., via a user activating the switch manually or via automatic activation of the switch when the testing device 101 is in use, such as when the cap is attached to the assembly). In some aspects, the light source may be at an angle relative to the test matrix, such as about 30° to about 60° relative to the test matrix, such as 45°. The light source may improve visualization of the test indicator lines and control lines of the test matrix, and may allow for visualization of analyte concentrations of about 0.01 picograms of analyte per 1 mL of fluid or greater. For example, the light source may be a light emitting diode (LED) tuned to emit light at a specific wavelength or wavelength range, such as light in a range having a peak wavelength of 660 nm. The wavelength of light that is emitted by the light source 12 may depend on the type of biomarker being measured and/or the detection particles used to visualize the biomarker. Additionally, while saliva has been presented herein as an example fluid for testing with the testing device 101, the testing device 101 may be adapted to receive and test other fluids, such as different biological samples (e.g., urine, serum, plasma, blood) or environmental samples (e.g., natural fluids and industrial plant effluents).

FIG. 5 shows a testing device 500 according to another embodiment of the disclosure. The testing device 500 includes a cap 502 and an assembly 510. Similar to the testing device 101, the testing device 500 is configured to collect a fluid (e.g., saliva) via the cap 502 and flow the collected fluid over one or more lateral flow strips housed in the assembly 510, as explained below. FIG. 5 shows the cap 502 in face-sharing connection with the assembly 510.

The cap 502 may be similar to the cap 2 of FIGS. 1-4 and may include a fluid collection device 504 and a sensor 506. In some examples, the cap 502 may have a different shape than the cap 2 of FIGS. 1-4, but may include an open end via which fluid may be collected.

The assembly 510 includes a sealing interface 512 coupled to a filter 514. The filter 514 may be the same as or similar to the filter 5 described above, e.g., a micropore filter. The assembly 510 may lack a fluid channel and the filter 514 may be directly coupled to one or more lateral flow strips. As shown, the assembly 510 includes a first lateral flow strip 516 and a second lateral flow strip 518, which may be separated by an internal wall 520. The first lateral flow strip 516 may be coupled between a first outer wall 530 and the internal wall 520, while the second lateral flow strip 518 may be coupled between a second outer wall 532 and the internal wall 520. Each lateral flow strip may include one or more test lines and a control line. For example, the first lateral flow strip 516 may include a first test line 522 and a first control line 524, and the second lateral flow strip 518 may include a second test line 526 and a second control line 528. Each test line may include antibodies specific to a respective analyte of interest, and each test line may test for a different analyte of interest. For example, the first test line 522 may include anti-E-cadherin antibodies and the second test line 526 may include anti-EGF antibodies.

FIG. 6 shows an example cap 600 that includes a funnel-shaped fluid collection device 602, e.g., a funnel-shaped collection funnel. The cap 600 may include threads or other fastening mechanisms to allow the cap 600 to be screwed on or otherwise coupled to a corresponding assembly at a filter 604 of the assembly. Cap 600 may be coupled to a suitable assembly, such as assembly 11 or assembly 510. In some examples, cap 2 and/or cap 502 may include a funnel-shaped collection device, as shown in FIG. 6. In some examples, the assembly (e.g., assembly 11 and/or assembly 510) may include a shape-matched sealing interface (e.g., complimentary to the shape of the fluid collection device) and thus in some examples the sealing interface may be complimentary to the funnel-shaped collection device.

A still further embodiment of a testing device 701 is shown in FIG. 7. Testing device 701 is similar to testing device 101, but the fluid collection device 3 in the testing device 701 may be integrated with the assembly 11 rather than be coupled to/integrated with the cap 2. In this way, the sample fluid may be collected via the first end 31 of the fluid collection device 3 and may flow directly to the filter 5, fluid channel 6, and test matrix 8. The cap 2 may be coupleable to the assembly 11 at the first end 31 of the fluid collection device 3. Coupling the cap 2 to the assembly 11 after fluid is collected in the fluid collection device 3 may cause pressure to be applied to the fluid, fluid collection device 3, filter 5, etc., which may increase the speed of the filtering of the fluid and wicking of the fluid across the test matrix 8. The amount of pressure applied by coupling the cap 2 to the assembly 11 may be about 20-70 psi, 30-60 psi, for example, about 40 psi, in an axial (e.g., longitudinal) direction (along the x axis in FIG. 7). By including the fluid collection device as part of the assembly, prevention of back flow of fluid as well as prevention of breakage of the filter may be provided.

In some examples, the lateral flow strips of the testing devices described herein may include gold nanoparticle (AuNP)-antibody, iridium oxide nanoparticle-antibody, and/or magnetite nanoparticle-antibody conjugates primed by adding polyclonal and or monoclonal antibodies. While such antibodies may be added in any amount useful for the intended purpose, in some aspects about 20-100 μl of polyclonal antibody and/or monoclonal antibody (40-300 μg/mL in phosphate buffered saline (PBS)) may be added into a solution of about 2-20 mL of AuNP with about 10-200 nm diameter. In some aspects, the AuNP may be bound to silica nanorods. The AuNP solution may be have any pH conducive to binding. In some examples, the solution may have a pH 7-9. The resulting conjugate may be suspended in a buffer solution, for example, but not limited to, 1-4% borate buffer at 4° C. until integrated. The use of such AuNP-silica nanorods, magnetite nanoparticles, or iridium oxide nanoparticles may achieve a 50 to 200 fold lower detection limit of biomarker levels in fluids as compared to without nanoparticles.

The lateral flow strips included in the testing devices described herein may be prepared by treating with various buffers at a pH of 7-8. For example, the sample and absorption pads or cellulose filter pad or other pads may be treated with one or more of borate buffer, Tween-20, or PBS in varying amounts and concentrations such as, but not limited to, being treated with 20-60 mM of 2% borate buffer, 0.05-0.1% Tween-20, the conjugate pad being treated with 2% borate buffer with 30-60 mM with a pH 7-8 with 8-20% sucrose and 0.02-0.1% Tween-20, and the nitrocellulose membrane being treated with PBS with 5-15 mM with a pH 7-8. After treatment, the pads may be dried at 37° C. In some examples, drying may take from 30-90 minutes or any fraction thereof.

The lateral flow strips may be generated by creating a test line area of any size desired. In some aspects, the size may be 5-10 mm² or any fraction thereof. For example, the test line area may be created via dispensing 0.5-2.5 μL of monoclonal or polyclonal antibody (such as anti-pepsin, Anti-E-cadherin, anti-TGF-α, anti-Epidermal Growth Factor, anti-IL-6, anti-MMP-2, anti-MMP-7 antibody, 40-300 μg/mL in PBS) on nitrocellulose membrane. A control line of the desired size may be created via dispensing 0.5-2.5 μL of secondary antibody such as goat, mouse anti-rabbit IgG antibody in 60-90 μg/mL in PBS. While the control line and test line may be separated by any distance desired, in some aspects, they are about 1-1.5 cm apart or any fraction thereof. The functional pads with the test and control lines may then be incubated, blocked and dried using methods known to those of skill in the art. For example, these lines may be incubated for about 20-90 minutes at 37° C., blocked with 100-300 μl of BSA for 10-30 minutes at room temperature, washed with PBS, and then dried for 4-10 hours at room temperature.

In some embodiments, the testing matrix may include a plurality of sections, as shown in FIG. 8. As illustrated, a testing matrix 800 includes a first section D1 comprising a sample collecting surface, a second section D2 including a filter, a third section D3 including a surface having one or more recognition agents which have specific affinity to a respective analyte or equivalent thereof linked to biomarkers or biosignatures (e.g., the label-agent conjugates discussed above), wherein the recognition agent generates a chemically and/or a physically detectable reaction. In this way, the third section D3 may include one or more test lines, each specific to a different analyte. The testing matrix may further include a fourth section D4 that includes a control line, where the control line may include a surface functionalized with an analyte or equivalent) e.g., a label antibody or other binding agent conjugate/analyte complex is captured by another antibody or other suitable binding agents which is primary to the analyte thereof) and/or a secondary antibody, and a fifth section D5 including a surface with a substrate molecule deposited thereon e.g., a zone or area whereas excess labeled antibody conjugate or other suitable binding agents will be captured by secondary antibody or other suitable binding agents, and a sixth section D6 including a surface for holding excess sample, wherein the first through sixth sections are arranged along a horizontal axis (e.g., the x-axis of FIG. 8) in an overlapping manner and in fluid communication, allowing lateral flow from the first section throughout all sections to the sixth section. In one embodiment, each section may overlap an adjacent section by an amount in the range of 0.1% to 100% of the total surface of the section.

The conjugate pad may be produced by loading about 10-40 μl of AuNP-antibody conjugate onto the entire pad and drying it. For example, the pad may be dried for 1-4 hours or any fraction thereof at room temperature. In some aspects, the sample and absorption pads may have an area of about 0.6-2.0 cm×0.9-2.5 cm. The end of each pad may be positioned to overlap each other for proper and smooth flow of sample.

The analytical performance of lateral flow strips of E-cadherin, TGF-α, EGF, IL-6, MMP-2, MMP-7 and pepsin was assessed using artificial saliva samples containing different concentrations of pepsin (0.01, 0.5, 1.0, 2.5, 5.0 pg/mL and 0.01, 0.5, 1.0, 2.5, 5.0 ng/mL in PBS) by mixing with 2-5% BSA, 0.001-0.01% Tween-20, and 0.5-2% methanol. 50-200 μL of each sample was placed onto the sample pad of the prepared immunochromatographic strip and allowed to flow for 5-20 minutes as explained in further detail in the Examples below. The colorimetric signal generated by the immuno-reaction was captured by using a digital camera or light with wavelength 350-700 nm. The blocking agents, buffers, and/or detergents described above are exemplary, and other buffers, blocking agents, or detergents may be used without departing from the scope of this disclosure (e.g., other buffers in the pH range described above may be used, such as Tris-buffered saline).

An advantage of the testing devices described herein is that test results may be provided within a few minutes, such as within about 5 minutes or less of applying the fluid to the assembly, or up to about 10 minutes after applying the fluid to the assembly. The testing devices described herein may be single-use, disposable devices, which may allow for the testing devices to be deployed at point-of-care, in-home, in the field, or virtually any location, and may allow for non-medical personnel to use the testing devices (e.g., patients), which may allow for rapid identification of the level of the analytes of interest and improve patient care. In other aspects, the testing devices may be re-usable.

In some aspects, the testing device may be integrated with a fast, rapid, and reliable image analysis system. For example, the image analysis system may include instructions stored in memory that are executable (e.g., by a processor) to analyze one or more images of the test matrix/lateral flow strips described herein in order to provide qualitative (e.g., disease state) or quantitative (e.g., biomarker concentration) test results. The instructions may be stored and executed on a mobile device (e.g., smartphone), a remote server, or a combination of the mobile device and the remote server. The image analysis system may analyze an image captured by a camera as explained above. In some examples, the image analysis system may be executed to analyze an image according to a method 900 shown in FIG. 9. Method 900 includes capturing images at 902. Capturing images may include capturing images based on interactive parameters definition, test parameters, and others suitable factors. For example, upon applying a fluid sample to the testing device, a user may position the camera of the mobile device and capture an image of the testing matrix, and the image may be saved in memory of the mobile device and/or sent to the remote server. In some examples, aspects of the camera (e.g., zoom, focus, exposure, etc.) may be controlled automatically to ensure a high quality image of the test line(s) and control line(s) is captured. At 904, method 900 includes performing data retrieval, which may include reading the captured image, processing the captured image, and reading data from the processed image. For example, the captured image may be processed to have a certain level of contrast, brightness, etc. At 906, method 900 includes identifying an area of interest of the image. The area of interest may include the test line(s) and control line(s). Once the area of interest is identified, the area of interest may be segmented (e.g., the other areas of the image may be cropped out of the image). Further, in some examples, a separate image may be formed for each test line and control line. To facilitate the identification of the area of interest in the image and further processing, the image may be rotated, point of care test data from the image may be extracted, a fine alignment of the image may be performed, and/or an orientation of the image may be adjusted. At 908, method 900 includes performing a segmentation of the image. For example, the test line(s) and control line(s) may be identified based on increased pixel intensity at the test line(s) and control line(s) relative to areas around the test line(s) and control line(s), and the identified areas of the image corresponding to each test line(s) and control line(s) may be segmented for further analysis. The segmentation and extraction may include applying a mask to each image and thresholding using the mask, such that points/pixels in the image that have a first channel to second channel (e.g., red to green channel) intensity ratio value that is higher than a threshold are maintained (e.g., set to 1) while other pixels are thresholded out (e.g., set to 0) (Foysal, K. H.; Seo, S. E.; Kim, M. J.; Kwon, O. S.; Chong, J. W. Analyte Quantity Detection from Lateral Flow Assay Using a Smartphone. Sensors 2019, 19, 4812. https://doi.org/10.3390/s19214812). At 910, a test line quantification is performed. The test line quantification may include calculating a median profile, estimating noise, estimating and subtracting background, detecting and testing line biomarkers or signatures, integrating a test line signal, assigning a test line, and/or quantifying the signal. For example, after segmenting the test line(s) and control line(s), background noise may be estimated based on an average pixel intensity of one or more regions outside of the test line(s) and control line(s). The estimated background noise may be subtracted from the pixel intensities of the test line(s) and control line(s). Then, the mean/average pixel intensity of each of the test line(s) and control line(s) may be determined. Alternatively, the background noise may be subtracted from the mean/average pixel intensity of the test line(s) and control line(s). Once the test line(s) and control line(s) have been background-corrected, the mean pixel intensities may be used to quantify the test line(s). For example, the control line may include the biomarker being tested by the test line at a known concentration, and the mean pixel intensity of the test line may be compared to the mean pixel intensity of the control line to determine a concentration of the biomarker in the tested fluid.

In some examples, the test and control lines in the image may be normalized to a standard blank background image (e.g., an image of the lateral flow strip before a sample is applied to the lateral flow strip), followed by selecting a specific wavelength for analysis (Foysal, K. H.; Seo, S. E.; Kim, M. J.; Kwon, O. S.; Chong, J. W. Analyte Quantity Detection from Lateral Flow Assay Using a Smartphone. Sensors 2019, 19, 4812. https://doi.org/10.3390/s19214812). Subsequently, selected areas of the test and control lines may be segmented via an automated algorithm and a local background may be taken in a round shape outside of the segmented area, and a mean pixel intensity of the local background may be subtracted from the average pixel intensity of the segmented spot and normalized to the sum of all the background subtracted spot signals (Belushkin A, Yesilkoy F, Altug H. Nanoparticle-Enhanced Plasmonic Biosensor for Digital Biomarker Detection in a Microarray. ACS Nano. 2018 May 22; 12(5):4453-4461. doi: 10.1021/acsnano.8b00519. Epub 2018 May 8. PMID: 29715005; herein after Belushkin).

In some examples, more than one area may be selected and segmented per test line and control line. In other examples, one area per test line and control line may be selected, where each area encompasses a majority or an entirety of the respective test line or control line. In some examples, the selected areas, after background subtraction, are analyzed to determine a disease state or condition. The disease conditions may include a relative likelihood the disease is present in the individual being tested (e.g., high likelihood, medium likelihood, low likelihood) or a stage of the disease (e.g., no disease, early stage, intermediate stage, advanced stage). Disease conditions (e.g., possible results from the test) may be ranked based on an iterative elimination method where the intensity of the area(s) of the test line is compared to the intensity of the area(s) of the control line, a probability is assigned to each possible disease based on the comparison, the disease conditions are ranked based on the probabilities, and any disease conditions that have a probability that meet a condition relative to a threshold may be eliminated, and the results may be cross validated using level concentration, where the ground absolute biomarker concentration is plotted against the predicted biomarkers concentration . In some aspects, intensities may be compared to previously determined reference levels, where varying intensities may indicate the presence or absence of a particular biomarker, or the presence or absence of a particular stage or condition, for example the stage of a specific disease.

Hereinafter, the present invention will be described in detail with reference to examples. It is to be understood that the terminology employed herein is used for the purpose of describing particular embodiments only and not to limit the scope of the invention.

### EXAMPLES

### Example 1

### Salivary Detection of E-cadherin, TGF-α, EGF, IL-6, MMP-2, MMP-7, and Pepsin

Lateral flow immunochromatographic strips for salivary detection of E-cadherin, TGF-α, EGF, IL-6, MMP-2, MMP-7, and pepsin were prepared. Gold nanoparticles (AuNPs, Sigma-Aldrich, St. Louis, MO, USA) were used for colorimetric label. The gold nanoparticle (AuNP)-antibody conjugate was primed by adding 50 μl of polyclonal antibody or monoclonal antibody (rabbit anti-pepsin, Anti-E-cadherin, anti-TGF-α, anti-EGF, anti-IL-6, anti-MMP-2, anti-MMP-7 antibody, 120 μg/mL in phosphate buffered saline (PBS)) into 10 mL of AuNP with 40 nm diameter solution with a pH 8, with 0.1M K₂CO₃ in dynamic stirring for 20-100 minutes at room temperature, and then desolated with 4 mL of bovine Serum Albumin (BSA) (6% in PBS). After 15 minutes the AuNP-antibody conjugate was centrifuged at 5000 rpm for 15 minutes and washing with borate buffer with a concentration of 3 mM with pH 7, two to four times. The resulting conjugate was suspended in a 3% borate buffer at 4° C. until integrated. Difference in AuNP size binding with antibody was analyzed by using a UV-Vis spectrophotometer. The colorimetric performance of the AuNP-antibody conjugate was analyzed by ELISA assays.

The lateral flow strip includes a sample pad, an absorption pad, a nitrocellulose membrane, and a conjugate pad. The sample and absorption pads or cellulose filter pad or other pads (Whatman Inc., Florham Park, NJ, USA) were treated with borate buffer with 40 mM and a pH 7 with 0.06% Tween-20 (Sigma-Aldrich, St. Louis, MO, USA). The conjugate pad was treated with 2% borate buffer with 40 mM with a pH 7 with 15% sucrose and 0.02% Tween-20. The nitrocellulose membrane was treated with PBS with 10 mM with a pH 7. After drying at 37° C. for 45 minutes, at least six functional membrane pads were kept in a desiccator at room temperature to avoid moisture contamination.

The treated functional pads were used to prepare an immunochromatographic strip. The test line area of 6 mm² was created via dispensing 2.0 μL of monoclonal or polyclonal antibody (anti-pepsin, Anti-E-cadherin, anti-TGF-α, anti-Epidermal Growth Factor, anti-IL-6, anti-MMP-2, anti-MMP-7 antibody, 240 μg/mL in PBS) on a nitrocellulose membrane (0.4 cm×2.5 cm). The control line was created via dispensing 1.5 μL of secondary antibody such as goat, mouse anti-rabbit IgG antibody in 80 μg/mL in PBS and separated from the test line via 1.2 cm. These lines were incubated for 20-90 minutes at 37° C., blocked with 200 μl of BSA for 20 minutes at room temperature, washed with PBS, and then dried for 6 hours at room temperature.

The conjugate pad with an area of 0.6 cm×0.6 cm was produced by loading 20 μL of AuNP-antibody conjugate onto the entire pad and drying it for 1 hour at room temperature. The sample and absorption pads had an area of 1.2 cm×1.2 cm. The end of each pad was positioned to overlap each other for proper and smooth flow of sample. The analytical performance of lateral flow strips of E-cadherin, TGF-α, EGF, IL-6, MMP-2, MMP-7 and pepsin was assessed using artificial saliva samples containing 1.0 ng/mL of pepsin in PBS by mixing with 3% BSA, 0.001% Tween-20, and 1.5% methanol. 100 μL of each sample was placed onto the sample pad of the prepared immunochromatographic strip and allowed to flow for 10 minutes. The colorimetric signal generated by the immuno-reaction was captured by using a digital camera or light with wavelength 350 and 660 nm.

### Example 2

### Silver Nanoparticle Generation

5 mL of as-prepared silver nanoparticle (AgNP) solution is centrifuged at 7000 rpm for 10 min. The AgNP pellet is resuspended with 10 mL of 0.1 M boric acid. Then, the pH of the AgNP solution is adjusted to pH 7.5 with 0.1 M sodium hydroxide. The desired anti-bodies, anti-pepsin, Anti-E-cadherin, anti-TGF-α, anti-Epidermal Growth Factor, anti-IL-6, anti-MMP-2, and anti-MMP-7 antibodies, are added dropwise to 1 mL AgNP solution to prepare the antibody-labeled AgNP. The AgNP is centrifuged at 7000 rpm for 10 min after incubation at room temperature for 2 h on a rolling mixer. The pellet is resuspended with 1 mL of 0.1 M borate buffer containing 0.1% w/v BSA (pH 7.5). The above solution is centrifuged at 7000 rpm for 10 min after 5 min incubation. The as-prepared AgNP is also resuspended with 0.1 M borate buffer to OD450 value at 1.5 and integrated into a lateral flow strip.

### Example 3

### Biomarker Levels

Saliva samples were taken from a plurality of subjects from 10 subjects by a drooling method and the testing device described herein. At least 10 minutes prior to collection of unstimulated saliva samples, subjects were asked to rinse orally with water and were asked to relax for 5-15 minutes. They were then seated in a bent forward position in an ordinary chair and were asked to put their tongues on the lingual surfaces of the upper incisors and to allow the saliva to drip into the fluid collection device of the testing device, which was treated with 50 g of 2% sodium azide solution, to prevent microbial decomposition of saliva. The devices were held to the lower lip for 1 to 2 minutes resulting in a collection of 1-5 mL of saliva per individual. Salivary pepsin and EGF levels were measured with ELISA kits. EGF was measured by using an ELISA kit from Quantikine R, R&D Systems Inc., USA and pepsin wasare measured by using the human pepsin ELISA kit (CUSABIO, HuBei, China). Data were analyzed by using Statistical Package for the Social Sciences (SPSS version 22; IBM Corporation, Armonk, NY). Table 1 shows the results of the mean and one standard deviation of the levels of the biomarkers by using drooling method and the testing device.

TABLE 1

| | Drooling | |
|---|---|---|
| Biomarkers | method | Testing device |
| EGF (pg/mL) | 453.2 (93.7) | 448.2 (99.2) |
| pepsin (ng/mL) | 162.3 (34.2) | 155.6 (24.2) |

Thus, no significant differences were found in the biomarker levels when fluid was collected using the drooling method or when fluid was collected using the testing device.

### Example 4

### Evaluation of the Lower Limit of Detection of Different Biomarker Levels

The 10 saliva samples from normal healthy individuals were taken as described above in Examples 1 and 3. Sample selection was done to ensure a wide range of concentrations to be measured with a testing device with nanoparticles (as described in Example 2) and a testing device without nanoparticles. Salivary biomarkers E-cadherin, TGF-α, EGF, MMP-2, MMP-7, and pepsin were measured by using the testing devices with integration of nanoparticles and without nanoparticle. The captured biomarkers were evaluated with 350 nm and 660 nm light sources. The wavelength of 350 and 660 light emitting diode (LED) source (Thorlabs) in combination with a bandpass filter with 350 and 660 nm center wavelength, respectively, and 10 nm full width at-half-maximum is used for narrow-band illumination at the wavelength of peak. The images were taken by using a 50× objective (Nikon). Image acquisition and processing was done using custom Matlab functions and a graphical user interface from a laptop connected to the CMOS camera as described in Belushkin. To determine detection cutoff times, signal from the test line and the control line and its 95% confidence interval were estimated at each time point. Detection limits are shown in Table 2.

TABLE 2
Detection limit of biomarkers

| | Limit of detection | |
|---|---|---|
| | Without | |
| | nanoparticle | With nanoparticle |
| E-cadherin | 1 | 0.01 |
| (pg/ml) | | |
| EGF (pg/mL) | 2.5 | 0.02 |
| TGF-α (pg/mL) | 0.2 | 0.001 |
| pepsin (pg/mL) | 0.01 | 0.005 |
| MMP-7 (pg/mL) | 0.1 | 0.001 |
| MMP-2 (pg/mL) | 0.2 | |

Thus, the testing device demonstrates an ultralow limit of detection (LOD) of 0.001 pg/mL of biomarker levels with nanoparticles, which is lower than the detection limit of the testing device without nanoparticles.

### Example 5

### Effect of Different Wavelength of Light on Biomarkers Levels

Saliva samples were taken as described in Example 4. The salivary TGF-α levels were measured with 350 nm and 660 nm light sources. The wavelength of 350 and 660 light emitting diode (LED) source (Thorlabs) in combination with a bandpass filter with 350 and 660 nm center wavelength, respectively, and 10 nm full width at-half-maximum is used for narrow-band illumination at the wavelength of peak. The images were taken by using a 50× objective (Nikon). Image acquisition and processing was done using custom Matlab functions and a graphical user interface from a laptop connected to the CMOS camera as described in Belushkin. To determine detection cutoff times, signal from the test line and the control line and its 95% confidence interval were estimated at each time point. A graph 1000 is shown in FIG. 10, which plots the signal intensity for each subject when measured using 350 nm light and 660 nm light.

As shown in FIG. 10, a 660 nm light source improved detection levels as compared to 330 nm.

The disclosure also provides support for a testing device, comprising: a fluid collection device adapted to collect a fluid, and an assembly including a filter coupled to a test matrix, the test matrix including one or more lateral flow strips adapted to optically indicate a level of one or more analytes in the fluid, where the fluid collection device is configured to supply the fluid to the one or more lateral flow strips via the filter. In a first example of the system, the system further comprises: a cap that is removably coupleable to the assembly via the fluid collection device. In a second example of the system, optionally including the first example, the fluid collection device is integrated with the cap, wherein the assembly further comprises a sealing interface at a collection end of the assembly, the sealing interface adapted to interface with the fluid collection device, wherein the sealing interface is coupled to the filter, and wherein at least a portion of the filter is exposed to atmosphere when the cap is not coupled to the assembly. In a third example of the system, optionally including one or both of the first and second examples, when the cap is coupled to the assembly, the at least the portion of the filter is fluidly coupled to an interior of the fluid collection device. In a fourth example of the system, optionally including one or more or each of the first through third examples, the sealing interface has a shape that is complimentary to a shape of the fluid collection device. In a fifth example of the system, optionally including one or more or each of the first through fourth examples, the fluid collection device is integrated with the assembly. In a sixth example of the system, optionally including one or more or each of the first through fifth examples, the fluid collection device is funnel-shaped. In a seventh example of the system, optionally including one or more or each of the first through sixth examples, the assembly further comprises a fluid channel fluidly coupled between the filter and the test matrix. In an eighth example of the system, optionally including one or more or each of the first through seventh examples, the fluid channel is configured to divide the fluid equally to each lateral flow strip. In a ninth example of the system, optionally including one or more or each of the first through eighth examples, the one or more lateral flow strips each comprise a plurality of sections arranged along a horizontal in an overlapping manner and in fluid communication. In a tenth example of the system, optionally including one or more or each of the first through ninth examples, each section the plurality of sections overlaps an adjacent section by an amount in a range of 0.1% to 100% of a total surface of the section. In a eleventh example of the system, optionally including one or more or each of the first through tenth examples, the system further comprises: a computing device configured to analyze an image of a test line and a control line of the one or more lateral flow strips, wherein the analyzing includes normalizing the test line and the control line to a standard blank background image, segmenting selected areas via an automated algorithm, identifying a local background comprising a round shaped outside of the segmented areas, subtracting a mean pixel intensity of the local background from an average pixel intensity of each segmented area, and normalizing to a sum of all the background subtracted area signals. In a twelfth example of the system, optionally including one or more or each of the first through eleventh examples, an average pixel intensity of one or more areas of the test line is compared to an average pixel intensity of one or more areas of the control line to identify a disease condition. In a thirteenth example of the system, optionally including one or more or each of the first through twelfth examples, the fluid collection device is adapted to collect saliva, and wherein the one or more lateral flow strips are adapted to optically indicate a level of one or more of E-cadherin, transforming growth factor-alpha (TGF-α), epidermal growth factor (EGF), interleukin-6 (IL-6), matrix metalloproteinase-2 (MMP-2), matrix metalloproteinase-7 (MMP-7), and pepsin in the saliva. In a fourteenth example of the system, optionally including one or more or each of the first through thirteenth examples, the testing device includes a data storing system which is configured to communicate with a reader and/or a computing device. In a fifteenth example of the system, optionally including one or more or each of the first through fourteenth examples, the filter has a pore size of about 0.01 μm to 2000 nm. In a sixteenth example of the system, optionally including one or more or each of the first through fifteenth examples, the fluid collection device includes a pad to collect the fluid with an indicator to indicate when sufficient fluid has been collected for analysis. In a seventeenth example of the system, optionally including one or more or each of the first through sixteenth examples, the system further comprises: an onboard light source to facilitate optical indication of a level of one or more analytes in the fluid, the onboard light source comprising UV light, IR light, ALI light, and/or visible light. In an eighteenth example of the system, optionally including one or more or each of the first through seventeenth examples, the onboard light source is configured to emit light at an angle of about 30° to about 60° relative to the test matrix. In a nineteenth example of the system, optionally including one or more or each of the first through eighteenth examples, the one or more lateral flow strips include a combination of nanoparticle and antibodies or antigens that facilitates visualization of analytes in the fluid at analyte concentrations of about 0.01 picogram of analyte per 1 milliliter fluid.

References to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. References to “about” when used together with a value, mean the value +/−10%, preferably about 5%, especially 1% of the value.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A testing device, comprising:
a fluid collection device adapted to collect a fluid; and
an assembly including a filter coupled to a test matrix, the test matrix including one or more lateral flow strips adapted to optically indicate a level of one or more analytes in the fluid, where the fluid collection device is configured to supply the fluid to the one or more lateral flow strips via the filter.

2. The testing device of claim 1, further comprising a cap that is removably coupleable to the assembly via the fluid collection device.

3. The testing device of claim 2, wherein the fluid collection device is integrated with the cap, wherein the assembly further comprises a sealing interface at a collection end of the assembly, the sealing interface adapted to interface with the fluid collection device, wherein the sealing interface is coupled to the filter, and wherein at least a portion of the filter is exposed to atmosphere when the cap is not coupled to the assembly.

4. The testing device of claim 3, wherein when the cap is coupled to the assembly, the portion of the filter is fluidly coupled to an interior of the fluid collection device.

5. The testing device of claim 4, wherein the sealing interface has a shape that is complimentary to a shape of the fluid collection device.

6. The testing device of claim 2, wherein the fluid collection device is integrated with the assembly.

7. The testing device of claim 1, wherein the fluid collection device is funnel-shaped.

8. The testing device of claim 1, wherein the assembly further comprises a fluid channel fluidly coupled between the filter and the test matrix.

9. The testing device of claim 8, wherein the fluid channel is configured to divide the fluid equally between each lateral flow strip.

10. The testing device of claim 1, wherein the one or more lateral flow strips each comprise a plurality of sections arranged along a horizontal axis in an overlapping manner and in fluid communication.

11. The testing device of claim 10, wherein each section the plurality of sections overlaps an adjacent section by an amount in a range of 0.1% to 100% of a total surface of the section.

12. The testing device of claim 1, further comprising a computing device configured to analyze an image of a test line and a control line of the one or more lateral flow strips, wherein the analyzing includes normalizing the test line and the control line to a standard blank background image, segmenting selected areas via an automated algorithm, identifying a local background comprising a round shaped outside of the segmented areas, subtracting a mean pixel intensity of the local background from an average pixel intensity of each segmented area, and normalizing to a sum of all the background subtracted area signals.

13. The testing device of claim 12, wherein an average pixel intensity of one or more areas of the test line is compared to an average pixel intensity of one or more areas of the control line to identify a disease condition.

14. The testing device of claim 1, wherein the fluid collection device is adapted to collect saliva, and wherein the one or more lateral flow strips are adapted to optically indicate a level of one or more of E-cadherin, transforming growth factor-alpha (TGF-α), epidermal growth factor (EGF), interleukin-6 (IL-6), matrix metalloproteinase-2 (MMP-2), matrix metalloproteinase-7 (MMP-7), and pepsin in the saliva.

15. The testing device of claim 1, wherein the testing device includes a data storing system which is configured to communicate with a reader and/or a computing device.

16. The testing device of claim 1, wherein the filter has a pore size of about 0.01 μm to 2000 nm.

17. The testing device of claim 1, wherein the fluid collection device includes a pad to collect the fluid, wherein the pad comprises an indicator to indicate when sufficient fluid has been collected for analysis.

18. The testing device of claim 1, further comprising an onboard light source to facilitate optical indication of a level of one or more analytes in the fluid, the onboard light source comprising UV light, IR light, ALI light, and/or visible light.

19. The testing device of claim 18, wherein the onboard light source is configured to emit light at an angle of about 30° to about 60° relative to the test matrix.

20. The testing device of claim 1, wherein the one or more lateral flow strips include a combination of nanoparticles coupled to antibodies or antigens, wherein the combination of nanoparticles coupled to antibodies or antigens facilitates visualization of analytes in the fluid at analyte concentrations of about 0.01 picogram of analyte per 1 milliliter fluid.
